(19) United States
(12) Patent Application Publication
(10) Pub. No.: US 2025/0295575 A1
(43) Pub. Date: Sep. 25, 2025

(54) COMPOSITION FOR DYEING HAIR

(71) Applicant: LG H&H CO., LTD., Seoul (KR)

(72) Inventors: Ju Hyeon JEON, Seoul (KR); Byung Tack LIM, Seoul (KR); Tae Hyung HEO, Seoul (KR); Seong Kil SON, Seoul (KR); Nae Gyu KANG, Seoul (KR); Hong Gu LEE, Seoul (KR)

(73) Assignee: LG H&H CO., LTD., Seoul (KR)

(21) Appl. No.: 18/860,504

(22) PCT Filed: Apr. 27, 2023
(86) PCT No.: PCT/KR2023/005775

§ 371 Date: Oct. 25, 2024

(30) Foreign Application Priority Data
Apr. 28, 2022 (KR) 10-2022-0052969
Nov. 16, 2022 (KR) 10-2022-0153749

(51) Int. Cl. A61K 8/97; A61Q 5/06

(52) U.S. Cl. CPC A61K 8/97; A61Q 5/065; A61K 2800/43; A61K 2800/592; A61K 2800/95

(57) ABSTRACT

The present invention relates to a hair cosmetic composition that assists in applying color to hair or maintaining the color of dyed hair. The hair cosmetic composition according to the present invention uses a cationic polymer compound having a specific structure, an amphoteric surfactant or a cationic surfactant, and a hair penetrating solvent, and thus can improve the color development and color retention of dyes compared to existing hair cosmetic compositions. In addition, through the synergistic effect of a combination of Terminalia chebula fruit extract and a basic amino acid, the hair color development can be increased, and the tensile strength, gloss, and friction coefficient of the hair can be remarkably improved.

### TECHNICAL FIELD

The present invention relates to a hair cosmetic composition that helps improve hair dyeing and maintain color.

### BACKGROUND ART

In general, compositions that change hair color can be divided into permanent hair dyes (oxidative hair dyes), semi-permanent hair dyes (non-oxidative hair dyes), and temporary hair dyes (powder compositions).

Permanent hair dyes have excellent color development but cause problems such as damage to the hair and scalp due to oxidation and allergies. Meanwhile, temporary hair dyes have insufficient color development and color retention due to the use of powder. On the other hand, semi-permanent hair dyes use dye to develop color, and compared to oxidative hair dyes, they do not cause damage or allergies. These semi-permanent hair dyes mainly use acid or basic dyes for coloring. However, semi-permanent hair dyes using dyes have low color development compared to existing oxidative hair dyes, so attempts have been made to overcome this problem.

However, when using a metal salt (U.S. Pat. No. 10,398,635), color development is improved, but due to metals, the composition has a negative effect on the sense of touch when used. When using polymers to enhance color development (Korean Unexamined Patent Publication No. 10-2016-0179033), basic dyes do not have sufficient improvement effects.

### DISCLOSURE

### Technical Problem

The present invention aims to provide a hair cosmetic composition that helps enhance hair dyeing and maintain the color of dyed hair.

In addition, the present invention aims to provide a composition for hair dyeing with improved hair tensile strength, gloss, or friction coefficients.

### Technical Solution

The present invention provides a hair cosmetic composition including:
(a) a basic dye;
(b) one or more cationic polymer compounds selected from the group consisting of polyquaternium-6, polyquaternium-7, polyethyleneimine or a derivative thereof, and polylysine or a derivative thereof;
(c) an amphoteric surfactant or a cationic surfactant; and
(d) a hair penetrating solvent, or
(a) a basic dye;
(e) Terminalia chebula fruit extract; and
(f) a basic amino acid.

### Advantageous Effects

The hair cosmetic composition according to the present invention further includes a cationic polymer compound; an amphoteric surfactant or a cationic surfactant; and a hair penetrating solvent, or further includes Terminalia chebula fruit extract and a basic amino acid, in a hair cosmetic composition including a basic dye. Thus, the color development (hair dyeing) and the color retention of dyes can be improved compared to existing hair cosmetic compositions including a basic dye.

In addition, the hair cosmetic composition according to the present invention can improve the tensile strength, gloss, and friction coefficient of the hair.

Furthermore, the hair cosmetic composition according to the present invention can exhibit the above-described effects without affecting the feeling of use and other factors.

### BEST MODES OF THE INVENTION

The present invention relates to a hair cosmetic composition including:
(a) a basic dye;
(b) one or more cationic polymer compounds selected from the group consisting of polyquaternium-6, polyquaternium-7, polyethyleneimine or a derivative thereof, and polylysine or a derivative thereof;
(c) an amphoteric surfactant or a cationic surfactant; and
(d) a hair penetrating solvent, or
(a) a basic dye;
(e) Terminalia chebula fruit extract; and
(f) a basic amino acid.

Hereinafter, the composition of the present invention will be described in detail.

When a part is said to “include” a certain component, this does not mean that other components are excluded, but it means that other components can be added, unless specifically stated to the contrary.

In the present invention, “dyeing” means applying dyes to the hair to change the inherent color of the surface, impart an intended color, and maintain the color.

In the present invention, the hair cosmetic composition may include (a) a basic dye; (b) a cationic polymer compound; (c) an amphoteric surfactant or a cationic surfactant; and (d) a hair penetrating solvent.

In the present invention, (a) the basic dye is a non-oxidative dye used as a semi-permanent or a temporary hair dye. Non-oxidative dyes can bind to the hair and develop color by interacting with various functional groups (such as —NH₂, —COOH, —SH, —OH, etc.) present inside and outside the hair.

Among them, a basic dye is cationized in an aqueous solution due to its amine group in the molecule. The cationized amine group can be fixed to the hair by being adsorbed (dyed) through ionic bonding with the minus portion of the keratin protein on the hair surface, which has a negative charge, or by partially penetrating into the hair. In other words, the basic dye may enhance color development by strengthening the adsorption mechanism of the dye to the hair surface and the penetration mechanism of the dye into the hair.

In a specific example, any compound used as a basic dye in the art may be used as the basic dye without limitation. For example, one or more selected from the group consisting of Basic Blue 7 (C.I. 42595), Basic Blue 16 (C.I. 12210), Basic Blue 22 (C.I. 61512), Basic Blue 26 (C.I. 44045), Basic Blue 99 (C.I. 56059), Basic Blue 117, Basic Violet 10 (C.I. 45170), Basic Violet 14 (C.I. 42515), Basic Brown 16 (C.I. 12250), Basic Brown 17 (C.I. 12251), Basic Red 2 (C.I. 50240), Basic Red 12 (C.I. 48070), Basic Red 22 (C.I. 11055), Basic Red 51, Basic Red 76 (C.I. 12245), Basic Red 118 (C.I. 12251:1), Basic Orange 31, Basic Yellow 28 (C.I. 48054), Basic Yellow 57 (C.I. 12719), Basic Yellow 87, Basic Black 2 (C.I. 11825), etc. may be used, or one or more selected from the group consisting of Basic Blue 3, Basic Blue 6, Basic Blue 7, Basic Blue 9, Basic Blue 26, Basic Blue 41, Basic Blue 47, Basic Blue 99, Basic Brown 4, Basic Brown 16, Basic Brown 17, Basic Green 1, Basic Green 4, Basic Orange 1, Basic Orange 2, Basic Orange 31, Basic Red 1, Basic Red 1:1, Basic Red 2, Basic Red 22, Basic Red 46, Basic Red 51, Basic Red 76, Basic Red 118, Basic Violet 1, Basic Violet 2, Basic Violet 3, Basic Violet 4, Basic Violet 10, Basic Violet 11:1, Basic Violet 14, Basic Violet 16, Basic Yellow 11, Basic Yellow 28, Basic Yellow 40, Basic Yellow 57, Basic Yellow 87, etc. may be used. Preferably, Basic Blue 99 and/or Basic Brown 16 may be used as the basic dye.

In a specific example, the content of (a) the basic dye is not particularly limited and may be 0.0001 to 5 wt % based on the total weight of the composition. Preferably, the basic dye may be included in an amount of 0.01 to 3 wt % or 0.1 to 1 wt %. When the content is low, there may be limitations in effective performance, and when the content is high, there may be problems in the formulation and the stabilization of the formulation.

In the present invention, (b) the cationic polymer compound may act as a hair surface coating agent when applied to the dyed hair to enhance dye fixation. Specifically, cationic polymer compounds may induce more active interactions between dyes and the hair by changing the state of functional groups present in the hair. Thus, it is possible to reduce the color fading of the dyed hair due to washing.

The cationic polymer compound may be one or more selected from the group consisting of polyquaternium-6, polyquaternium-7, polyethyleneimine or a derivative thereof, and polylysine or a derivative thereof. The cationic polymer compound may preferably be polyquaternium-6.

In general, when a basic dye and a cationic polymer having the same ionicity are treated simultaneously, the two components compete with each other, interfering with the dye's adsorption to the hair. Therefore, it may reduce the color development of the basic dye. However, the cationic polymer compound according to the present invention does not act as a competitor of basic dyes due to its structural characteristics even when simultaneously treated with basic dyes. The cationic polymer compound may be helpful for the color development of the dye by acting as a coating agent that enhances the fixation of basic dyes.

Polyquaternium-6, polyquaternium-7, polyethyleneimine or a derivative thereof, and polylysine or a derivative thereof according to the present invention have a high degree of repeated positive charges and a high cationization degree. Since the positive charge is fixed within or close to the main chain molecule, the rigidity of the charge is relatively high. Therefore, the cationic polymer compound may act as an excellent film forming agent. In addition, the cationic polymer compound may have an excellent coating ability for the negatively charged hair surface and enhance the fixation of the dye.

In a specific example, the content of (b) the cationic polymer compound is not particularly limited and may be 0.0001 to 5 wt % based on the total weight of the composition. Preferably, the cationic polymer compound may be included in an amount of 0.001 to 3 wt % or 0.01 to 2 wt %. When the content of the cationic polymer compound is too high, the polymer is applied to the hair first, thereby hindering the adsorption or penetration of the basic dye. On the other hand, when the content is too low to reach an effective concentration, it is impossible to realize the fixation of the basic dye. In other words, within the above-described content range, the interaction between the functional group of the hair and the dye may be actively promoted.

In the present invention, (c) the amphoteric surfactant or the cationic surfactant may be applied with a basic dye and a cationic polymer compound to further enhance the hair adsorption and hair penetration of the basic dye in a cleansing formulation.

In a specific example, the amphoteric surfactant may include one or more selected from the group consisting of coco-betaine, cocamidopropyl betaine, lauramidopropyl betaine, cocoamphocarboxyglycinate, cocoamphodipropionate, cocoamphodiacetate, laurylhydroxysultaine, and salts thereof.

In a specific example, the cationic surfactant may be a quaternary ammonium and/or a tertiary amido amine. The quaternary ammonium may include one or more selected from the group consisting of behentrimonium chloride, cetrimonium chloride, steartrimonium chloride, dicetyldimonium chloride, and distearyldimonium chloride, and the tertiary amido amine may include one or more selected from the group consisting of cocamidopropyldimethylamine, stearamidopropyldimethylamine, and behenamidopropyldimethylamine.

In a specific example, the content of (c) the amphoteric surfactant or cationic surfactant is not particularly limited and may be 0.01 to 30 wt % based on the total weight of the composition. Preferably, the amphoteric surfactant or cationic surfactant may be included in an amount of 0.1 to 20 wt % or 1 to 15 wt %. When the content is excessive, the transfer of basic dyes to the hair may be hindered, and when the content is low, it may not act as a hair cleanser due to its insufficient cleansing ability.

In the present invention, (d) the hair penetrating solvent may help the basic dye penetrate into the hair more easily, making it easier for the hair and the basic dye to bond.

In a specific example, the hair penetrating solvent may be an alcohol. The alcohol may be, for example, one or more selected from the group consisting of ethanol, propyl alcohol, isopropyl alcohol, propylene glycol, butylene glycol, methylpropanediol, hexylene glycol, diethylene glycol, dipropylene glycol, benzyl alcohol, and glycerin. Preferably, the hair penetrating solvent may include benzyl alcohol.

In a specific example, the content of (d) the hair penetrating solvent is not particularly limited and may be 0.0001 to 15 wt % based on the total weight of the composition. Preferably, the hair penetrating solvent may be included in an amount of 0.01 to 15 wt % or 0.1 to 10 wt %. When the content is excessive, the feeling of use may be reduced due to serious damage to the hair, scalp irritation, or a severe odor, and when the content is low, it may be impossible to achieve the effect of helping the basic dye penetrate into the hair more easily. When the hair penetrating solvent with the above-described content is applied, the solubility of the basic dye may be improved, and thus the dye may penetrate into the hair more easily.

In the present invention, the hair cosmetic composition may include (a) a basic dye, (b) a cationic polymer compound, (c-1) an amphoteric surfactant, and (d) a hair penetrating solvent, and the weight ratio of (a):(b):(c-1):(d) may be 0.01 to 1:0.001 to 1:1 to 25:0.01 to 15.

In the present invention, the hair cosmetic composition may include (a) a basic dye, (b) a cationic polymer compound, (c-2) a cationic surfactant, and (d) a hair penetrating solvent, and the weight ratio of (a):(b):(c-2):(d) may be 0.01 to 1:0.001 to 1:0.01 to 10:0.01 to 15.

In the present invention, the hair cosmetic composition may include (a) a basic dye; (e) Terminalia chebula fruit extract; and (f) a basic amino acid.

In the present invention, (a) the basic dye may be one of the above-described types, and preferably, Basic Brown 16, Basic Blue 99, Basic Yellow 87, and/or Basic Red 51 may be used as the basic dye.

In a specific example, the content of (a) the basic dye is not particularly limited and may be 0.001 to 5 wt % based on the total weight of the composition. Preferably, the basic dye may be included in an amount of 0.01 to 2 wt % or 0.05 to 1 wt %. When the content is too high, the dye may easily stain the skin or cause problems in the stability of the formulation, and when the content is low, the effect of improving color development may not be achieved.

In the present invention, (e) the Terminalia chebula fruit extract is an extract obtained by hot water extraction or alcohol extraction of the natural material, the fruit of Terminalia chebula.

The Terminalia chebula fruit extract may strongly fix the basic dye to the hair and improve color development. In addition, since the Terminalia chebula fruit extract includes a large amount of anionic polyphenols composed of chebulic acid, etc., when applied to the hair with a cationic basic dye, the two substances with counter ions mutually act as cross-linking agents for the hair, allowing the basic dye to be more strongly fixed to the hair surface. In addition, a synergistic coloring effect may be achieved because the polyphenols are dyed on the hair surface without using a separate metal salt.

In a specific example, the Terminalia chebula fruit extract may be a hot water extract of the Terminalia chebula fruit. The Terminalia chebula fruit extract extracted through hot water extraction may impart a better dyeing effect compared to the alcohol extract.

In a specific example, the content of (e) the Terminalia chebula fruit extract is not particularly limited and may be 0.0001 to 5 wt % based on the total weight of the composition. Preferably, the Terminalia chebula fruit extract may be included in an amount of 0.0001 to 5 wt % or 0.0001 to 1 wt %. When the content is low, the adhesion of the basic dye may be reduced due to insufficient interaction with the basic dye, and when the content is high, the stability of the formulation may be reduced.

In the present invention, (f) the basic amino acid may have a positive charge at neutral pH. The basic amino acid may impart an additional mordant coloring effect to the hair cosmetic composition while increasing the tensile strength and gloss of the hair. In general, metal salts have been used for the mordant coloring of polyphenols, but the metal salts make the hair surface very rough, reducing the feeling of use. The present invention may enhance color development of the hair and improve the tensile strength, gloss, and friction coefficient of the hair by excluding metal salts commonly used in dyeing with natural dyes and using a basic amino acid.

In a specific example, the basic amino acid may include one or more selected from the group consisting of arginine, lysine, histidine and salts thereof.

In a specific example, the basic amino acid may be a peptide or polypeptide, which includes the basic amino acid as a component, and may include, for example, an arginine/lysine polypeptide.

In a specific example, the content of (f) the basic amino acid is not particularly limited and may be 0.0001 wt % or more based on the total weight of the composition. Preferably, the basic amino acid may be included in an amount of 0.0001 to 1 wt % or 0.0001 to 0.5 wt %. When the content is low, the effect of improving the color development, tensile strength, gloss, and friction coefficient of the hair is insignificant, and when the content is high, the amino acid precipitates, which may reduce the stability of the formulation.

In the present invention, the hair cosmetic composition includes (a) a basic dye, (e) Terminalia chebula fruit extract, and (f) a basic amino acid, and the weight ratio of (e):(a):(f) may be 1:0.001 to 1:0.001 to 1. It may preferably be 1:0.005 to 0.5:0.005 to 0.5, more preferably, 1:0.01 to 0.1:0.01 to 0.1 (Terminalia chebula fruit extract:basic dye:basic amino acid). Within the above weight ratio range of the three components, the effect of improving the color development, tensile strength, gloss, and friction coefficient of the hair may be significantly enhanced.

In a specific example, the hair cosmetic composition including (a) a basic dye, (e) Terminalia chebula fruit extract, and (f) a basic amino acid may have a pH of 3 to 11, preferably 3.5 to 10.5.

In a specific example, a metal salt may not be substantially included in the hair cosmetic composition including (a) a basic dye, (e) Terminalia chebula fruit extract, and (f) a basic amino acid. Preferably, the metal salt may be included in an amount of less than 1 wt %, less than 0.1 wt %, or less than 0.01 wt % based on the total weight of the composition, or may not be included at all. The hair cosmetic composition of the present invention does not substantially include a metal salt, thereby lowering the friction coefficient of the hair and making the hair surface smooth.

In a specific example, an amphoteric amino acid may not be substantially included in the hair cosmetic composition including (a) a basic dye, (e) Terminalia chebula fruit extract, and (f) a basic amino acid. Preferably, the amphoteric amino acid may be included in an amount of less than 1 wt %, less than 0.1 wt %, or less than 0.01 wt % based on the total weight of the composition, or may not be included at all. Amphoteric amino acids may reduce the binding force of a basic dye to the hair by hindering the interaction between the basic dye and Terminalia chebula fruit extract. Thus, the hair cosmetic composition of the present invention may prevent this problem because amphoteric amino acids are not substantially included.

The hair cosmetic composition of the present invention may additionally include substances that are commonly used in hair cosmetics. Specifically, the hair cosmetic composition may additionally include components commonly used in hair dyes within a range that does not reduce the effects of the present invention. The hair cosmetic composition may include appropriately selected components such as dyes, solvents, water-soluble moisturizers, alkaline agents, antioxidants, metal ion sequestrants, pH control agents, thickeners, or fragrances.

The hair cosmetic composition of the present invention may be applied to hair products. Such hair products may be, for example, pre-shampoo compositions, shampoos, coloring shampoos, rinses, treatments, waxes, gels, sprays, mousses, hair lotions, hair packs, hair essences, hair creams, permanent hair dyes, temporary hair dyes, perm agents, non-woven fabrics, sheets, and the like.

In a specific example, the hair cosmetic composition may be a semi-permanent hair dye as a non-oxidative hair dye.

In a specific example, the hair cosmetic composition may be a hair cleansing composition or a hair care composition, and preferably a hair cleansing composition. The hair cleansing composition or the hair care composition may be an expression which excludes hair dyes using oxidative agents.

In a specific example, the hair cosmetic composition may be applied to a hair cosmetic composition in a wash-off form. Even when the hair cosmetic composition is applied to a hair cosmetic composition in a wash-off form, the effects of improving color development and maintaining the color may be maintained or enhanced.

All components described in the present invention preferably do not exceed each maximum usage amount specified in the relevant laws and regulations of Korea, China, the United States, Europe, Japan, etc. (such as Regulations on Safety Standards for Cosmetics (Korea) and Safety and Technical Standards for Cosmetics (China)). In other words, the hair cosmetic composition according to the present invention includes the components according to the present invention within an amount permitted by the relevant laws and regulations of each country.

In addition, the present invention provides a hair treatment method including a step of applying the hair cosmetic composition according to the present invention to the hair.

In the present invention, after dyeing the hair using the hair cosmetic composition, the color difference value (ΔE) of the hair before and after dyeing, which is measured using a colorimeter for color measurement, may be 20 or more. The color difference value (ΔE) of the hair may be measured according to the method of Experimental Example 1, and preferably, ΔE may be 30 to 80, and more preferably, 50 to 80, or 55 to 80.

The color difference value may be calculated using HunterLab's Agera Spectrophotometer according to Equation 1 below.

$$Δ E = {[ {( {L}_{0}^{*} - {L}_{1}^{*} )}^{2} + {( {a}_{0}^{*} - {a}_{1}^{*} )}^{2} + {( {b}_{0}^{*} - {b}_{1}^{*} )}^{2} ]}^{1 / 2} \qquad [ Equation 1 ]$$

In Equation 1, L* represents lightness and darkness, and the higher the number, the brighter it is. a is the green to red coordinate, and the higher the number, the stronger the redness. b* is the blue to yellow coordinate, and the higher the number, the stronger the yellowness. L₀*, a₀*, and b₀* are the color values before dyeing, and L₁*, a₁*, and b₁* are the color values after dyeing.

### BEST MODES OF THE INVENTION

Hereinafter, the present invention will be described in detail through examples. The following examples are intended to illustrate the present invention, and the scope of the present invention is not limited thereto. These examples are provided to complete the disclosure of the present invention and to fully inform those skilled in the art of the scope of the invention, and the present invention is defined by the scope of the claims. In addition, commercially available raw materials were purchased and used as the components used in the present invention.

### EXAMPLES

### Examples 1-1 to 1-8 and Comparative Examples 1-1 and 1-2

Basic Brown 16 (CI 12250) and Basic Blue 99 (CI 56059), which are basic dyes commonly prescribed for dyeing gray hair, were used as basic dyes. A dye solution was prepared by dissolving the two basic dyes in water in a 1:1 ratio at a concentration of 0.4% (w/w). In the dye solution, four cationic polymer compounds (polyquaternium-6, polyquaternium-7, polyethyleneimine, and polylysine), a surfactant, and a hair penetrating solvent were dissolved according to the content ratios shown in Tables 1 and 2 below.

TABLE 1

| | | Comparative | Comparative | | |
|---|---|---|---|---|---|
| | Component (wt %) | Example 1-1 | Example 1-2 | Example 1-1 | Example 1-2 |
| | Water | Appropriate | Appropriate | Appropriate | Appropriate |
| | | amount | amount | amount | amount |
| Dye | Basic Blue 99 | 0.2 | 0.2 | 0.2 | 0.2 |
| | Basic Brown 16 | 0.2 | 0.2 | 0.2 | 0.2 |
| Cationic | Polyquaternium-6 | — | 0.5 | 0.5 | — |
| polymer | Polyquaternium-7 | — | — | — | 0.5 |
| compound | Polyethyleneimine | — | — | — | — |
| | Polylysine | — | — | — | — |
| Anionic | Sodium laureth | — | 10 | — | — |
| surfactant | sulfate | | | | |
| Amphoteric | Cocamidopropyl | — | — | 10.5 | 10.5 |
| surfactant | betaine | | | | |
| Cationic | Behentrimonium | — | — | — | — |
| surfactant | chloride | | | | |
| Solvent | Benzyl alcohol | — | 20 | 0.5 | 0.5 |
| | Sum | 100 | 100 | 100 | 100 |

TABLE 2

| | | Example | Example | Example | Example | Example | Example |
|---|---|---|---|---|---|---|---|
| | Component (wt %) | 1-3 | 1-4 | 1-5 | 1-6 | 1-7 | 1-8 |
| | Water | Appropriate | Appropriate | Appropriate | Appropriate | Appropriate | Appropriate |
| | | amount | amount | amount | amount | amount | amount |
| Dye | Basic Blue 99 | 0.2 | 0.2 | 0.2 | 0.2 | 0.2 | 0.2 |
| | Basic Brown 16 | 0.2 | 0.2 | 0.2 | 0.2 | 0.2 | 0.2 |
| Cationic | Poly quaternium-6 | — | — | 0.5 | — | — | — |
| polymer | Poly quaternium-7 | — | — | — | 0.5 | — | — |
| compound | Polyethyleneimine | 0.5 | — | — | — | 0.5 | — |
| | Polylysine | — | 0.5 | — | — | — | 0.5 |
| Anionic | Sodium laureth | — | — | — | — | — | — |
| surfactant | sulfate | | | | | | |
| Amphoteric | Cocamidopropyl | 10.5 | 10.5 | — | — | — | — |
| surfactant | betaine | | | | | | |
| Cationic | Behentrimonium | — | — | 2.8 | 2.8 | 2.8 | 2.8 |
| surfactant | chloride | | | | | | |
| Solvent | Benzyl alcohol | 0.5 | 0.5 | 0.5 | 0.5 | 0.5 | 0.5 |
| | Sum | 100 | 100 | 100 | 100 | 100 | 100 |

### Experimental Example 1. Comparison of Effect of Enhancing Coloring

The degree to which the coloring is enhanced by adding a cationic polymer compound, surfactant, and a hair penetrating solvent to a basic dye was compared.

The experimental yak wool (Phoenix) was washed with a 15% sodium laureth sulfate (SLES) solution corresponding to 10% of the weight of the tresses, and then dried using a dryer. The initial color values (L₀*, a₀*, and b₀*) of the yak wool before dyeing were measured using a colorimeter (Agera spectrophotometer from HunterLab, light source D65/10).

The yak wool was soaked in 20 mL of each composition of Comparative Examples and Examples for 5 minutes. After some time, the dyed yak wool was washed in running lukewarm water (20 to 30° C.) for 1 minute. After wiping off the moisture, it was dried using a dryer, and the color values (L₁*, a₁*, and b₁*) of the dyed yak wool were measured using a colorimeter. The color values before and after dyeing were calculated, and the color difference (ΔE) value was calculated according to Equation 1 below.

$$Δ E = {[ {( {L}_{0}^{*} - {L}_{1}^{*} )}^{2} + {( {a}_{0}^{*} - {a}_{1}^{*} )}^{2} + {( {b}_{0}^{*} - {b}_{1}^{*} )}^{2} ]}^{1 / 2} \qquad [ Equation 1 ]$$

The measured color difference (ΔE) values are shown in Table 3 below.

TABLE 3

| | Compar- | Compar- | | | |
|---|---|---|---|---|---|
| Color | ative | ative | | | |
| develop- | Example | Example | Example | Example | Example |
| ment | 1-1 | 1-2 | 1-1 | 1-2 | 1-3 |
| ΔE | 65.49 | 14.34 | 56.17 | 55.87 | 55.90 |
| Color | | | | | |
| develop- | Example | Example | Example | Example | Example |
| ment | 1-4 | 1-5 | 1-6 | 1-7 | 1-8 |
| ΔE | 55.89 | 67.63 | 66.17 | 67.18 | 66.84 |

In Comparative Example 1-2 using the most commonly used anionic surfactant in hair cleansing formulations, the color development of the dye is significantly reduced because the adsorption to the hair and the penetration into the hair of the dye are hindered due to ionic interactions.

However, it is confirmed that when an amphoteric surfactant or a cationic surfactant is applied as in Example 1-1 or Example 1-5 and the content of the hair penetrating solvent is appropriately adjusted, color development is further enhanced. The same can be confirmed not only in Examples 1-1 or 1-5 using polyquaternium-6, but also in Examples 1-2 to 1-4 or Examples 1-6 to 1-8 using polyquaternium-7, polyethyleneimine, and polylysine.

Through this experimental example, it can be confirmed that when a cationic polymer compound, an amphoteric surfactant or a cationic surfactant, and a hair penetrating solvent are applied simultaneously and their contents are optimized, the penetration of the basic dye into the hair significantly increases while imparting a hair cleansing effect, and at the same time, a mechanism to prevent dye elution works through the formation of a polymer coating film on the hair surface, and thus a powerful dye locking effect is achieved.

### Example 2-1 and Comparative Examples 2-1 to 2-6

Example 2-1 and Comparative Examples 2-1 to 2-6 including a basic dye, Terminalia chebula fruit extract, and a basic amino acid (arginine/lysine polypeptide; Peptiskin®) were prepared according to the contents (wt %) shown in Table 4 below.

TABLE 4

| | Comparative | Comparative | Comparative | Comparative | Comparative | Comparative | |
|---|---|---|---|---|---|---|---|
| | Example | Example | Example | Example | Example | Example | Example |
| | 2-1 | 2-2 | 2-3 | 2-4 | 2-5 | 2-6 | 2-1 |
| Water | Appropriate | Appropriate | Appropriate | Appropriate | Appropriate | Appropriate | Appropriate |
| | amount | amount | amount | amount | amount | amount | amount |
| Cocamidopropyl | 10 | 10 | 10 | 10 | 10 | 10 | 10 |
| betaine | | | | | | | |
| Coco-betaine | 2.7 | 2.7 | 2.7 | 2.7 | 2.7 | 2.7 | 2.7 |
| Disodium | 1.7 | 1.7 | 1.7 | 1.7 | 1.7 | 1.7 | 1.7 |
| cocoamphodiacetate | | | | | | | |
| Glyceryl oleate | 0.625 | 0.625 | 0.625 | 0.625 | 0.625 | 0.625 | 0.625 |
| Coco-glucoside | 0.875 | 0.875 | 0.875 | 0.875 | 0.875 | 0.875 | 0.875 |
| Cocamide MEA | 1.2 | 1.2 | 1.2 | 1.2 | 1.2 | 1.2 | 1.2 |
| PEG-150 | 1.7 | 1.7 | 1.7 | 1.7 | 1.7 | 1.7 | 1.7 |
| Distearate | | | | | | | |
| EDTA 4NA | 0.5 | 0.5 | 0.5 | 0.5 | 0.5 | 0.5 | 0.5 |
| Citric acid | 0.15 | 0.15 | 0.15 | 0.15 | 0.15 | 0.15 | 0.15 |
| monohydrate | | | | | | | |
| Glycerin | 1 | 1 | 1 | 1 | 1 | 1 | 1 |
| Basic Brown 16 | 0.135 | — | — | 0.135 | 0.135 | — | 0.135 |
| Basic Blue 99 | 0.02 | — | — | 0.02 | 0.02 | — | 0.02 |
| Basic Yellow 87 | 0.08 | — | — | 0.08 | 0.08 | — | 0.08 |
| Basic Red 51 | 0.15 | — | — | 0.15 | 0.15 | — | 0.15 |
| Terminalia | — | 5 | — | 5 | — | 5 | 5 |
| chebula fruit | | | | | | | |
| extract | | | | | | | |
| Arginine/lysine | — | — | 0.5 | — | 0.5 | 0.5 | 0.5 |
| polypeptide | | | | | | | |
| Sum | 100 | 100 | 100 | 100 | 100 | 100 | 100 |

### Experimental Example 2. Comparison of Effect of Enhancing Coloring

The effect of enhancing coloring of Example and Comparative Examples was measured according to the method of Experimental Example 1.

The dyeing was performed by washing the yak wool with the composition one time and three times repeatedly. After the yak wool was soaked in water for washing, the prepared composition was applied in an amount corresponding to 10% of the weight of the yak wool tresses, washed with foam for 1 minute, left for 3 minutes, and then rinsed under running water for 1 minute. The ΔE measurement method is the same as in Experimental Example 1.

The measured color difference (ΔE) values are shown in Table 5 below.

TABLE 5

| | | Comparative | Comparative | Comparative | Comparative | Comparative | Comparative | |
|---|---|---|---|---|---|---|---|---|
| | | Example | Example | Example | Example | Example | Example | Example |
| | | 2-1 | 2-2 | 2-3 | 2-4 | 2-5 | 2-6 | 2-1 |
| ΔE | One- | 16.34 | 0.56 | 0.07 | 18.73 | 13.81 | 2.02 | 19.94 |
| | time | | | | | | | |
| | treatment | | | | | | | |
| | Three- | 23.91 | 0.62 | 0.19 | 29.15 | 19.89 | 5.83 | 32.56 |
| | time | | | | | | | |
| | treatment | | | | | | | |

When comparing Comparative Example 2-1 and Comparative Example 2-4, it can be seen that when Terminalia chebula fruit extract is additionally applied to the basic dye, the fixation of the basic dye is enhanced due to mutual ionic cross-linking between the two substances, and the ΔE value slightly increases due to the mordant coloring effect of polyphenols.

As in Example 2-1, when a basic amino acid is additionally applied to Comparative Example 2-4, it can be seen that color development is significantly enhanced. This is because the cationic basic amino acid enhances the mordant coloring of the polyphenols in the Terminalia chebula fruit extract, and the amount of the basic dye penetrating into the hair cortex increases due to the basic amino acid with a great ability to penetrate into the hair.

As the hair cosmetic composition treatment process is repeated (1 time to 3 times), the difference in ΔE value increases, and thus it is confirmed that it is possible to gradually realize natural dyeing when the hair cosmetic composition is used every day.

### Experimental Example 3. Evaluation of Hair Tensile Strength and Gloss

### (1) Preparation of Samples

A bleaching agent was applied in an amount twice the weight of the tresses to experimental Burex normal hair (prepared in bundles of 4 g each), sealed and left at room temperature for 20 minutes, and then washed with a 15% SLES solution for 1 minute and dried. The bleached hair prepared by the above method was washed with a regular shampoo (Debon Charming Shampoo was used) to prepare a control group. The tensile strength and gloss change rates of Comparative Examples 2-1 to 2-3 and Example 2-1 compared to the control group were measured.

### (2) Measurement of Tensile Strength

Hair samples were prepared by placing crimps at 30 mm intervals at both ends of one hair strand of the tresses from the control group, Comparative Examples 2-1 to 2-3 and Example 2-1 and pressing it with a press. The cross-sectional area of the hair was measured using a laser scan micrometer, and then the tensile strength of each hair strand after measuring cross-sectional area was measured using a tensile tester (MTT175 from Dia-Stron, UK). When a hair sample was placed on a holder and pulled at a tensile speed of 20 mm/min, the intensity of the force at which the hair breaks (break load) was calculated as the break load per hair area to evaluate hair tensile strength (break stress). The increase rate compared to the measured value of the control group was expressed in percentage (%).

### (3) Gloss Evaluation

The tresses of Comparative Examples 2-1 to 2-3 and Example 2-1 were aligned on a gloss measurement device (SAMBA Hair System from Bossa Nova Vision). The numerical value (BNT), which is comprehensively calculated from the reflection and scattering that occurs when the aligned hair surface is irradiated with polarized light and the width of the reflection band captured in images, was confirmed. The larger the value, the better the gloss. The increase rate compared to the measured value of the control group was expressed as a percentage (%).

TABLE 6

| | Comparative | Comparative | Comparative | Example |
|---|---|---|---|---|
| Increase rate (%) | Example 2-1 | Example 2-2 | Example 2-3 | 2-1 |
| Tensile strength | 4.91 | 6.19 | 12.19 | 18.33 |
| Gloss | 28.76 | 13.17 | 17.01 | 35.89 |

It is confirmed that compared to the control group which treated with a general shampoo, Comparative Examples 2-1, 2-2, and 2-3 which include a dye, Terminalia chebula fruit extract, and a basic amino acid, respectively, showed increases in tensile strength and gloss value. This is because the strength of the surface and inside of the hair is reinforced due to the hair surface being coated with the dye and polyphenols or the basic amino acid penetrating into the hair. In addition, the decrease in the brightness value due to dyeing of the tresses or the increase in nutrition due to extract treatment resulted in an increase in the gloss value.

Furthermore, in Example 2-1, which simultaneously included a dye, Terminalia chebula fruit extract, and a basic amino acid, the tensile strength and the gloss value were the highest. As the penetration of the basic dye and polyphenols is promoted due to the basic amino acid with a great ability to penetrate into the hair, the density of the hair cortex increases, and thus tensile strength is enhanced. In addition, the color becomes more vivid due to the reduction in the peeling of the dye which deeply penetrated into the hair, and the increase in the mordant coloring of polyphenols, and hair alignment increases due to the inherent conditioning effect of cationic amino acids, thereby increasing the measured gloss value.

### Experimental Example 4. Evaluation of Hair Friction Coefficient

Experimental Phoenix bleached hair (4 g) was prepared, and an untreated sample was used as a control group. The bleached hair was dyed by soaking it in the solutions prepared according to Comparative Examples 2-7 to 2-9 and Example 2-2 in Table 7 for 20 minutes. Afterward, a 15% SLES solution was applied in an amount corresponding to 10% of the weight of the tresses, washed with foam for 1 minute, rinsed in running water for 1 minute, towel-dried, and dried with a dryer for measurement.

TABLE 7

| | Comparative | Comparative | Comparative | Example |
|---|---|---|---|---|
| | Example 2-7 | Example 2-8 | Example 2-9 | 2-2 |
| Water | Appropriate | Appropriate | Appropriate | Appropriate |
| | amount | amount | amount | amount |
| Basic | 0.1 | 0.1 | 0.1 | 0.1 |
| Brown 16 | | | | |
| Terminalia | 5 | 5 | 5 | 5 |
| chebula fruit | | | | |
| extract | | | | |
| Ferric chloride | — | 0.5 | — | — |
| Zinc sulfate | — | — | 0.5 | — |
| Arginine/lysine | — | — | — | 0.5 |
| polypeptide | | | | |
| Sum | 100 | 100 | 100 | 100 |

The roots of the tresses of Comparative Examples 2-7 to 2-9 and Example 2-2 were bonded and fixed to an acrylic plate, and the friction coefficient was measured using a friction tester (MTT 175 from Dia-stron, UK). The friction coefficient was calculated by measuring the change in force that occurs when the fiber holder attached to the tester is moved over the hair surface at a constant speed under a constant normal force. Since a lower friction coefficient means a smoother hair surface, the greater the increase rate (%) of the friction coefficient, the rougher the hair surface becomes.

TABLE 8

| Increase | Comparative | Comparative | Comparative | Example |
|---|---|---|---|---|
| rate (%) | Example 2-7 | Example 2-8 | Example 2-9 | 2-2 |
| Friction | 17.07 | 75.61 | 63.41 | 4.88 |
| coefficient | | | | |

In Comparative Examples 2-8 and 2-9 which include metal salts, the friction coefficient of the hair is significantly increased with just one treatment. Through this result, it is confirmed that when metal salts are included in hair care compositions that are used every day, such as shampoos, the hair becomes much rougher with repeated use of the compositions.

On the other hand, in Example 2-2 including a basic amino acid instead of a metal salt, the friction coefficient is not significantly increased compared to the control group. In addition, compared to Comparative Example 2-7 which does not include a basic amino acid and includes only a dye and Terminalia chebula fruit extract, the friction coefficient is reduced as additional conditioning is provided due to the characteristics of cationic amino acids.

### INDUSTRIAL APPLICABILITY

The hair cosmetic composition according to the present invention can improve the color development (hair dyeing) and the color retention of dyes compared to existing hair cosmetic compositions including a basic dye.

In addition, the hair cosmetic composition according to the present invention can improve the tensile strength, gloss, and friction coefficient of the hair.

1. A hair cosmetic composition comprising:
(a) a basic dye;
(b) one or more cationic polymer compounds selected from the group consisting of polyquaternium-6, polyquaternium-7, polyethyleneimine or a derivative thereof, and polylysine or a derivative thereof;
(c) an amphoteric surfactant or a cationic surfactant; and
(d) a hair penetrating solvent, or
(a) a basic dye;
(e) Terminalia chebula fruit extract; and
(f) a basic amino acid.

2. The composition of claim 1, wherein the content of (a) the basic dye is 0.0001 to 5 wt % based on the total weight of the composition.

3. The composition of claim 1, wherein the content of (b) the cationic polymer compound is 0.0001 to 5 wt % based on the total weight of the composition.

4. The composition of claim 1, wherein the amphoteric surfactant is one or more selected from the group consisting of coco-betaine, cocamidopropyl betaine, lauramidopropyl betaine, cocoamphocarboxyglycinate, cocoamphodipropionate, cocoamphodiacetate, laurylhydroxysultaine, and salts thereof.

5. The composition of claim 1, wherein the cationic surfactant is one or more selected from the group consisting of behentrimonium chloride, cetrimonium chloride, steartrimonium chloride, dicetyl diimonium chloride, distearyldimonium chloride, cocamidopropyl dimethylamine, stearamidopropyldimethylamine, and behenamidopropyldimethylamine.

6. The composition of claim 1, wherein the content of (c) the amphoteric surfactant or the cationic surfactant is 0.01 to 30 wt % based on the total weight of the composition.

7. The composition of claim 1, wherein (d) the hair penetrating solvent includes one or more alcohols selected from the group consisting of ethanol, propyl alcohol, isopropyl alcohol, propylene glycol, butylene glycol, methylpropanediol, hexylene glycol, diethylene glycol, dipropylene glycol, benzyl alcohol, and glycerin, alone or in combination.

8. The composition of claim 1, wherein the content of (d) the hair penetrating solvent is 0.0001 to 15 wt % based on the total weight of the composition.

9. The composition of claim 1, wherein the weight ratio of the basic dye:cationic polymer compound:amphoteric surfactant:hair penetrating solvent is 0.01 to 1:0.001 to 1:1 to 25:0.01 to 15.

10. The composition of claim 1, wherein the weight ratio of the basic dye:cationic polymer compound:cationic surfactant:hair penetrating solvent is 0.01 to 1:0.001 to 1:0.01 to 10:0.01 to 15.

11. The composition of claim 1, wherein (e) the Terminalia chebula fruit extract is a hot water extract of the fruit of Terminalia chebula.

12. The composition of claim 1, wherein the content of (e) the Terminalia chebula fruit extract is 0.0001 to 5 wt % based on the total weight of the composition.

13. The composition of claim 1, wherein (f) the basic amino acid is one or more selected from the group consisting of arginine, lysine, histidine, and salts thereof.

14. The composition of claim 1, wherein the content of (f) the basic amino acid is 0.0001 to 1 wt % based on the total weight of the composition.

15. The composition of claim 1, wherein the weight ratio of the Terminalia chebula fruit extract:basic dye:basic amino acid is 1:0.001 to 1:0.001 to 1.

16. The composition of claim 1, wherein the composition including (a) a basic dye; (e) Terminalia chebula fruit extract; and (f) a basic amino acid has a pH of 3.5 to 10.5.

17. The composition of claim 1, wherein the composition including (a) a basic dye; (e) Terminalia chebula fruit extract; and (f) a basic amino acid does not include a metal salt and/or an amphoteric amino acid.
